(19) United States
(12) Patent Application Publication
(10) Pub. No.: US 2014/0343612 A1
(43) Pub. Date: Nov. 20, 2014

(54) SPINAL CORRECTION SYSTEM

(71) Applicant: Warsaw Orthopedic, Inc., Warsaw, IN (US)

(72) Inventors: William Alan Rezach, Atoka, TN (US); Charles Anthony Dickerson, Bartlet, TN (US)

(73) Assignee: Warsaw Orthopedic, Inc., Warsaw, IN (US)

(21) Appl. No.: 13/896,972

(22) Filed: May 17, 2013

(51) Int. Cl. A61B 17/70

(52) U.S. Cl. CPC A61B 17/7032; A61B 17/7056; A61B 17/7041
USPC 606/276

(57) ABSTRACT

A spinal construct comprises a longitudinal member extending between a first end and a second end and including an intermediate portion. A construct member includes a first surface and a second surface movable relative to the first surface in a configuration for connection with a first portion of vertebral tissue. The construct member defines an implant cavity configured for disposal of the first end. Systems and methods are disclosed.

### TECHNICAL FIELD

The present disclosure generally relates to medical devices for the treatment of musculoskeletal disorders, and more particularly to a surgical system for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. In some cases, post-surgical complications can result. These complications can include deformities, for example, junctional kyphosis, which may lead to spinal imbalance and progressive low back pain and stiffness. Correction treatments may employ implants that are manipulated for engagement with vertebrae to position and align one or more vertebrae. This disclosure describes an improvement over these prior art technologies.

### SUMMARY

In one embodiment, a spinal construct is provided. The spinal construct comprises a longitudinal member extending between a first end and a second end and includes an intermediate portion. A construct member includes a first surface and a second surface movable relative to the first surface in a configuration for connection with a first portion of vertebral tissue. The construct member defines an implant cavity configured for disposal of the first end. In some embodiments, systems and methods are provided.

### BRIEF DESCRIPTION OF THE DRAWINGS

The present disclosure will become more readily apparent from the specific description accompanied by the following drawings, in which:

FIG. 1 is a breakaway, perspective view of components of one embodiment of a system in accordance with the principles of the present disclosure;

FIG. 2 is a perspective view of components of the system shown in FIG. 1;

FIG. 3 is a plan view of the components shown in FIG. 2;

FIG. 4 is a breakaway, perspective view of components of one embodiment of a system in accordance with the principles of the present disclosure disposed with vertebrae, in part phantom;

FIG. 5 is a perspective view of components of one embodiment of a system in accordance with the principles of the present disclosure;

FIG. 6 is a plan view of the components shown in FIG. 5;

FIG. 7 is a perspective view of components of one embodiment of a system in accordance with the principles of the present disclosure;

FIG. 8 is a perspective view of the components shown in FIG. 7 with parts separated; and

FIG. 9 is a side cross section view of the components shown in FIG. 7.

### DETAILED DESCRIPTION

The exemplary embodiments of a surgical system and related methods of use are discussed in terms of medical devices for the treatment of musculoskeletal disorders and more particularly, in terms of a spinal correction system. In some embodiments, the spinal correction system may be employed in applications for correction of deformities, such as scoliosis and kyphosis. In some embodiments, the spinal correction system may be employed in applications for avoiding and/or treating post-surgical deformities, such as, for example, in patients who had previously undergone spinal surgery either for scoliosis or for degenerative low back conditions. In some embodiments, the spinal correction system may be employed in applications for treating flat back syndrome. In some embodiments, the spinal correction system may be employed in applications for avoiding and/or treating junctional kyphosis, such as, for example, proximal junctional kyphosis. In some embodiments, the spinal correction system may be employed in applications for treating an imbalance of the spine, such as, for example, a sagittal imbalance and progressive low back pain and stiffness.

In some embodiments, the present disclosure provides a spinal construct including a hook and claw construct. In some embodiments, the spinal construct includes a hook combination, which includes a pedicle hook up-going, and a lamina or transverse process down-going claw combination. In some embodiments, the spinal construct includes a hook construct that allows a spinal rod to stop at a vertebral level below an upper instrumented vertebral level, which may include a vertebral level one level below the upper instrumented vertebral level. For example, in one embodiment, the spinal construct includes an up going pedicle hook positioned at a thoracic vertebra T4 level and a down-going claw positioned at a thoracic vertebra T3 level. This configuration provides a spinal construct that spans two or more vertebral levels and/or bodies. This configuration allows mid-line structures of an upper instrumented vertebral level to remain intact. In some embodiments, the spinal construct allows a surgeon to transition stiffness to the upper instrumented vertebral level.

In some embodiments, the spinal construct includes setscrew locking that includes a coupling member. In some embodiments, the spinal construct includes a setscrew that tightens and locks an extension of the construct. In one embodiment, the setscrew is centered with a body of the construct. In one embodiment, the setscrew is biased to one side of the body. In one embodiment, the setscrew is biased to one side of the body and is offset from a longitudinal axis of an implant, such as, for example, a spinal rod, disposed with the body.

In some embodiments, the spinal construct includes a body and an extension connected to the body such that the extension laterally translates into a channel of the body. In some embodiments, the spinal construct includes a body and an extension connected to the body such that the extension axially translates through a closed channel of the body. In some embodiments, the spinal construct includes a body and an extension connected to the body such that the extension can rotate or spin to mate with anatomy and/or with the UIV level.

In some embodiments, the spinal construct includes a crown locking configuration. In some embodiments, the spinal construct includes a ratcheting type of compression across the extension for fixation.

In some embodiments, one or all of the components of the spinal correction system may be disposable, peel-pack, pre-packed sterile devices. One or all of the components of the system may be reusable. The system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

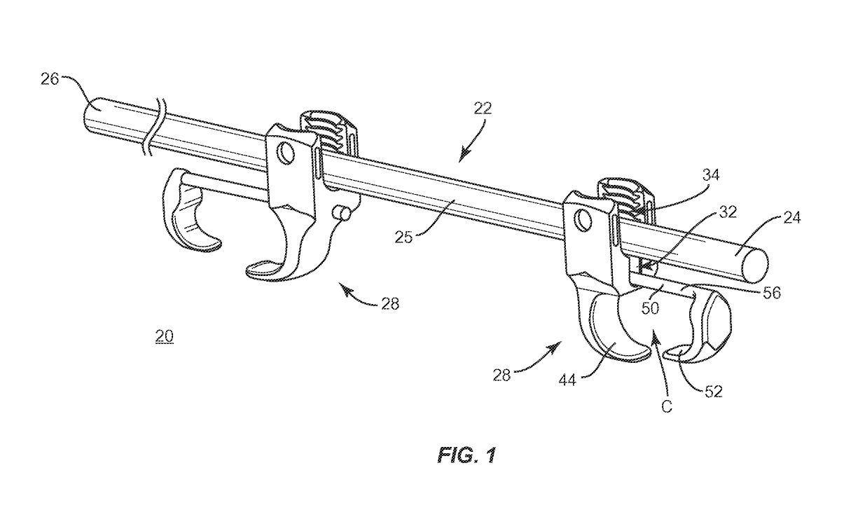

The following discussion includes a description of a spinal correction system, related components and methods of employing the surgical correction system in accordance with the principles of the present disclosure. Alternate embodiments are also disclosed. Reference will now be made in detail to the exemplary embodiments of the present disclosure, which are illustrated in the accompanying figures. Turning to FIGS. 1-3, there are illustrated components of a spinal correction system 20.

The components of spinal correction system 20 can be fabricated from biologically acceptable materials suitable for medical applications, including metals, synthetic polymers, ceramics, bone material, tissue and/or their composites, depending on the particular application and/or preference of a medical practitioner. For example, the components of spinal correction system 20, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of spinal correction system 20 may have material composites, including the above materials, to achieve various desired characteristics such as strength, rigidity, elasticity, compliance, biomechanical performance, durability and radiolucency or imaging preference. The components of spinal correction system 20, individually or collectively, may also be fabricated from a heterogeneous material such as a combination of two or more of the above-described materials. The components of spinal correction system 20 may be monolithically formed, integrally connected or include fastening elements and/or instruments, as described herein.

Spinal correction system 20 is employed, for example, with an open, mini-open or minimally invasive including percutaneous surgical technique to attach a longitudinal member to a spine that has a spinal disorder. Spinal correction system 20 includes a spinal construct comprising a construct member, such as, for example, a hook construct 28 that is employed with a longitudinal member, such as, for example, a spinal implant or vertebral rod 22. Rod 22 is substantially cylindrical and extends between a first end 24 and a second end 26 and includes an intermediate portion 25 disposed therebetween. In one embodiment, rod 22 is disposed to extend along an axial plane, such as for example, a sagittal plane of a body of a patient. The components of spinal correction system 20 may be employed in surgical correction procedures for avoiding and/or treating post-surgical deformities, such as, for example, proximal junctional kyphosis. In some embodiments, spinal correction system 20 may be employed in applications for treating an imbalance of a spine, such as, for example, a sagittal imbalance.

In some embodiments, spinal correction system 20 may include one or a plurality of longitudinal members. In some embodiments, one or all of a plurality of longitudinal members may be disposed in various relative orientations, such as, for example, side-by-side, parallel, transverse, perpendicular or angular and/or be disposed to extend along substantially coronal, sagittal and transverse planes of a body.

Rod 22 has a uniform thickness/diameter. In some embodiments, rod 22 may have various surface configurations, such as, for example, rough, threaded for connection with surgical instruments, arcuate, undulating, porous, semi-porous, dimpled, polished and/or textured according to the requirements of a particular application. In some embodiments, the thickness defined by rod 22 may be uniformly increasing or decreasing, or have alternate diameter dimensions along its length. In some embodiments, rod 22 may have various cross section configurations, such as, for example, oval, oblong, triangular, rectangular, square, polygonal, irregular, uniform, non-uniform, variable and/or tapered.

In some embodiments, rod 22 may have various lengths. In some embodiments, rod 22 may be made from autograft and/or allograft and be configured for resorbable or degradable applications. In one embodiment, rod 22 is a cadaver tendon. In one embodiment, rod 22 is a tendon that may be harvested, for example, from a patient or donor. In some embodiments, all or only a portion of rod 22 may have a semi-rigid, flexible or elastic configuration and/or have elastic and/or flexible properties similar to the properties from materials, such as, for example, fabric, silicone, polyurethane, silicone-polyurethane, copolymers, rubbers, polyolefin rubber, elastomers, thermoplastic elastomers, thermoset elastomers and elastomeric composites. In one embodiment, rod 22 provides a selective amount of expansion and/or extension in an axial direction. In some embodiments, rod 22 may have a flexible configuration, which includes movement in a lateral or side to side direction. In some embodiments, rod 22 may be compressible in an axial direction. Rod 22 can include a plurality of separately attachable or connectable portions or sections, such as bands or loops, or may be monolithically formed as a single continuous element.

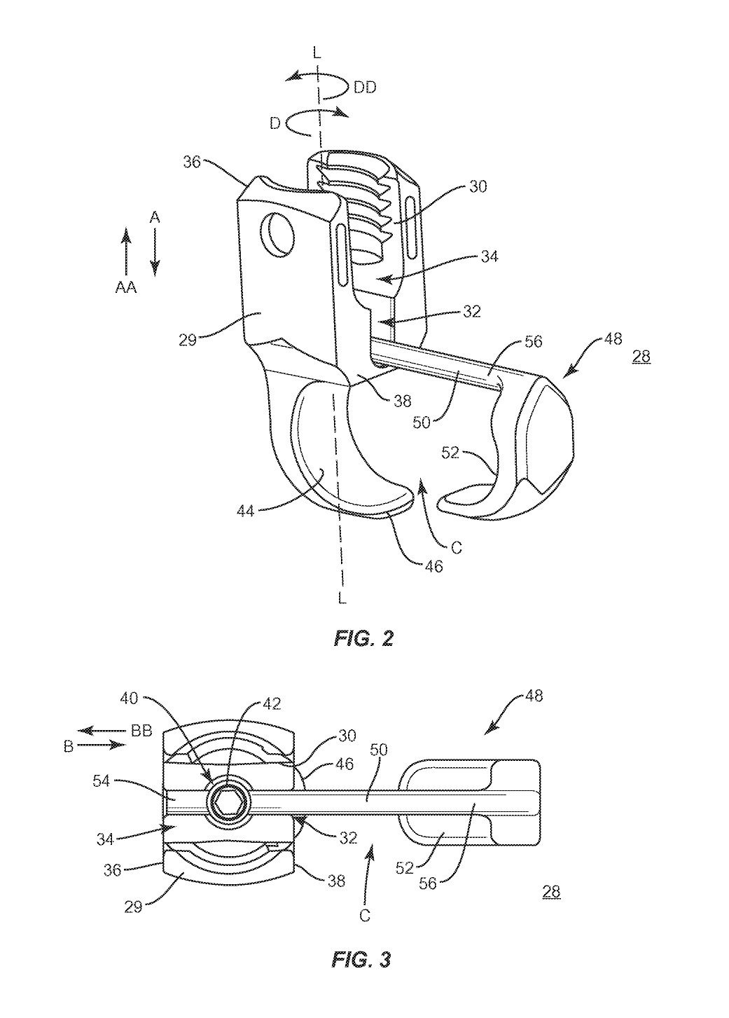

Hook construct 28 that defines a longitudinal axis L. Construct 28 includes a first surface, such as, for example, a body 29 including an inner surface 30. Body 29 includes a pair of arms that define a U-shaped cavity 34 extending parallel to axis L and configured for disposal of rod 22. Cavity 34 extends through a proximal end surface of body 29 such that rod 22 can be disposed with body 29 by inserting rod 22 through cavity 34, in the direction shown by arrow A in FIG. 2, until rod 22 engages surface 30. In some embodiments, all or only a portion of cavity 34 may have alternate cross section configurations, such as, for example, oval, oblong, triangular, square, polygonal, irregular, uniform, non-uniform, offset, staggered, and/or tapered. In some embodiments, cavity 34 may be disposed at alternate orientations, relative to axis L, such as, for example, transverse, perpendicular and/or other angular orientations such as acute or obtuse, co-axial and/or may be offset or staggered. In some embodiments, spinal correction system 20 may include one or a plurality of hook constructs. In some embodiments, cavity 34 extends through a side surface of body 29 without extending through the proximal end surface of body 29 such that rod 22 can be side loaded into cavity 34 by inserting rod into cavity 34, in the direction shown by arrows B, BB.

Cavity 34 is threaded and engageable with a coupling member, such as, for example, a setscrew (not shown) to fix rod 22 with body 29 relative to construct 28. In some embodiments, surface 30 can include a thread form located adjacent one of the arms and a thread form located adjacent the other arm, each thread form being configured for engagement with a setscrew.

Surface 30 defines a passageway 32 that communicates with cavity 34. Passageway 32 extends parallel to axis L and has an open, U-shaped configuration. In some embodiments, all or only a portion of passageway 32 may have alternate cross section configurations, such as, for example, oval, oblong, triangular, square, polygonal, irregular, uniform, non-uniform, offset, staggered, and/or tapered. In some embodiments, passageway 32 may be disposed at alternate orientations relative to axis L, such as, for example, transverse, perpendicular and/or other angular orientations relative to axis L, such as acute or obtuse, co-axial and/or may be offset or staggered.

Passageway 32 and cavity 34 each extend between and through an end surface 36 and an opposite end surface 38 of body 29. Passageway 32 and cavity 34 each have a length defined by the distance between surface 36 and surface 38. In some embodiments, passageway 32 is beveled adjacent surface 36 and/or surface 38 such that a width of passageway 32 increases adjacent surface 36 and/or surface 38 to facilitate insertion of a second surface, such as, for example, an extension 48 into passageway 32, in the direction shown by arrow BB in FIG. 2. In some embodiments, passageway 32 has a uniform diameter along the length of passageway 32. In some embodiments, passageway 32 is tapered between surface 36 and surface 38 and/or between surface 38 and surface 36.

Surface 30 defines a circular recess 40 extending parallel to axis L through a portion of passageway 32. Recess 40 has a width that is greater than a width of passageway 32 and is positioned at a midpoint between surface 36 and surface 38 along surface 30. Recess 40 includes a thread form configured to engage a thread form of a coupling member, such as, for example, a setscrew 42 to fix extension 48 within passageway 32. In some embodiments, a proximal portion of recess 40 is beveled to conform to a configuration of setscrew 42 such that a proximal end surface of setscrew 42 is flush with cavity 34 when the thread form on setscrew 42 engages the thread form in recess 40 or the proximal end surface of setscrew 42 is positioned distal to cavity 34 when the thread form on setscrew 42 engages the thread form in recess 40. In some embodiments, recess 40 is offset such that recess 40 is disposed closer to end 36 than end 38 or closer to end 38 than end 36. In some embodiments, recess 40 may be disposed at alternate orientations relative to axis L, such as, for example, transverse, perpendicular and/or other angular orientations relative to axis L, such as acute or obtuse, co-axial and/or may be offset or staggered. In some embodiments, all or only a portion of recess 40 may be variously configured and dimensioned, such as, for example, those alternatives described herein. In some embodiments, recess 40 may be fixed with setscrew 42 in alternate fixation configurations, such as, for example, friction fit, pressure fit, locking protrusion/recess, locking keyway and/or adhesive.

Body 29 includes a hook 44 extending laterally therefrom. Hook 44 is monolithically formed with body 29 and includes an inner surface having an arcuate configuration. The inner surface of hook 44 is oriented in a first direction such that the inner surface of hook 44 faces toward end 38. The inner surface of hook 44 has a radius of curvature such that a tip 46 of hook 44 extends beyond surface 38. In some embodiments, the inner surface of hook 44 has a radius of curvature such that tip 46 is disposed in substantial alignment with surface 38. In some embodiments, tip 46 is disposed below surface 38. In some embodiments, hook 44 is variously shaped, such as, for example, those alternatives described herein. In some embodiments, hook 44 is formed with body 29 via integral connection or can be attached. In some embodiments, hook 44 is removably engaged with the body of construct 28 via frictional engagement, threaded engagement, mutual grooves, screws, adhesive, nails or barbs.

Hook construct 28 includes extension 48, which comprises a post 50 and a hook 52 extending from post 50. Post 50 is translatable within passageway 32 and has a length extending between a first end 54 and a second end 56. The length of post 50 is greater than the length of passageway 32 such that end 54 may extend beyond surface 36 when post 50 is disposed within passageway 32. In some embodiments, post 50 has a length that spans multiple vertebral levels such as, for example, the distance between a first vertebra and a second vertebra adjacent the first vertebra such that hook 44 or hook 52 can engage the first vertebra and the other of hook 44 and hook 52 can engage the second vertebra. Post 50 has a cylindrical configuration that defines a uniform diameter along the length of post 50. Hook 52 extends from end 56 and includes an arcuate inner surface oriented in a second direction that faces toward the inner surface of hook 44. End 54 is disposed in passageway 32 and end 56 is spaced apart from passageway 32 when end 54 is disposed in passageway 32. Passageway 32 has a depth that is greater than the diameter of post 50 such that post 50 is spaced apart from cavity 34 when post 50 is disposed in passageway 32. In some embodiments, passageway 32 has a depth that is less than the diameter of post 50 such that at least a portion of post 50 extends cavity 34 when post 50 is disposed in passageway 32. In some embodiments, post 50 is variously shaped, such as, for example, those alternatives described herein. In some embodiments, an outer surface of post 50 includes surface configurations to enhance fixation with passageway 32, such as, for example, rough, arcuate, undulating, porous, semi-porous, dimpled, polished and/or textured. In some embodiments, hook 52 is variously shaped, such as, for example, those alternatives described herein. In one embodiment, hook 52 includes a claw configuration.

Hook 52 is movable relative to hook 44, in the direction shown by arrows B, BB in FIG. 3, such that the inner surfaces of hooks 44, 52 define an adjustable tissue cavity C for connection with a portion of tissue, such as, for example, vertebral tissue. Tissue cavity C is selectively adjustable about a configuration of the portion of tissue. In one embodiment, extension 48 is rotatable such that hook 52 is rotatable about the axis of post 50 and relative to body 29 to mate with the vertebral tissue. In some embodiments, post 50 is configured such that hook 52 is offset from hook 44 so that hook construct 28 can be connected with non-aligned vertebra and/or bony structures of the spine. In one embodiment, post 50 has an arcuate configuration such that hook 52 is offset from hook 44. In some embodiments, post 50 may include a staggered configuration such that hook 52 is offset from hook 44.

In some embodiments, spinal correction system 20 includes a kit such that the size of tissue cavity C may be varied by selecting a hook 44 having a selected radius of curvature, selecting a hook 52 having a selected radius of curvature and/or selecting a post 50 having a selected length. The radius of curvature of the inner surface of hook 44, the radius of curvature of the inner surface of hook 52 and/or the length of post 50 may therefore be adjusted to provide tissue cavity C with a size adapted for a particular application. In some embodiments, the radius of curvature of the inner surface of hook 52 is equal to the radius of curvature of the inner surface of hook 44 to enhance engagement with tissue. In some embodiments, the radius of curvature of the inner surface of hook 52 is less than the radius of curvature of the inner surface of hook 44 to enhance engagement with tissue.

Setscrew 42 is disposed in recess 40 while post 50 is positioned in passageway 32 to fix post 50 within passageway 32 and hook 44 relative to hook 52. Setscrew 42 is translatable in recess 40 along axis L, in the direction shown by arrows A, AA, by rotating setscrew 42, in the direction shown by arrows D, DD. Setscrew 42 is translated until a planar distal end surface of setscrew 42 engages an outer surface of post 50 to fix post 50 within passageway 32 and hook 44 relative to hook 52. In some embodiments, the distal end surface of setscrew 42 includes surface configurations to enhance engagement with the outer surface of post 50, such as, for example, those alternatives described herein. In some embodiments, the distal end surface of setscrew 42 includes a deformable material, such as, for example, silicone or silicone rubber configured to enhance engagement with the outer surface of post 50. In some embodiments, all or only a portion of the distal end surface of setscrew 42 may be variously configured and dimensioned, such as, for example, planar, concave, polygonal, irregular, uniform, non-uniform, staggered, tapered, consistent or variable.

In operation, a practitioner manipulates hook construct 28 for disposal about a vertebral level to fix and/or attach construct 28 with a targeted section of a spine. The targeted section of the spine can include a pedicle, transverse process and/or a lamina of a vertebral level such that hook construct 28 connects an implant, such as, for example, rod 22 with vertebrae.

In assembly, operation and use, a surgical system, including spinal correction system 20, similar to system 20 described herein, is employed with a surgical correction procedure. For example, spinal correction system 20 may be employed in surgical procedures for avoiding and/or treating post-surgical deformities, such as, for example, in patients who had previously undergone spinal surgery either for scoliosis or for degenerative low back conditions, such that one or a plurality of hook constructs and vertebral rods of system 20 are used for avoiding and/or treating proximal junctional kyphosis.

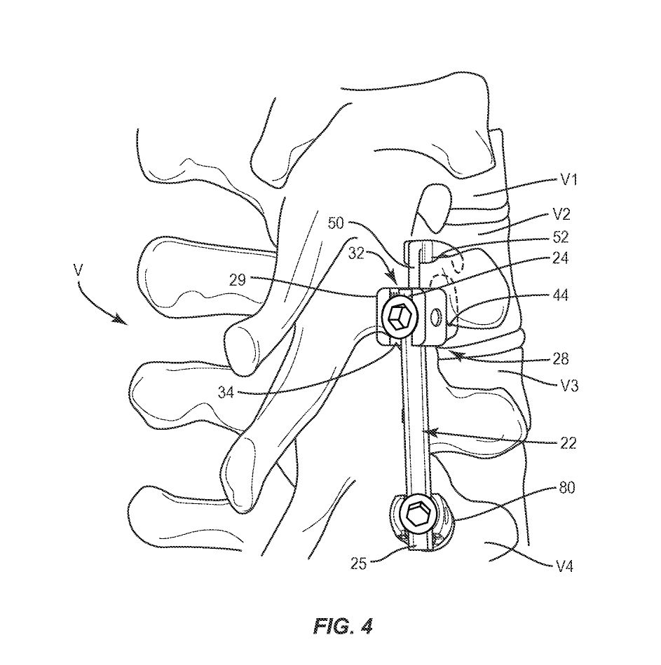

In some embodiments, one or all of the components of spinal correction system 20 can be delivered or implanted as a pre-assembled device or can be assembled in situ. The components of spinal correction system 20 may be completely or partially revised, removed or replaced. For example, spinal correction system 20 can be employed with a surgical correction treatment of an applicable condition or injury of an affected section of a spinal column and adjacent areas within a body, such as, for example, vertebrae V that includes vertebrae V1-V4, as shown in FIG. 4. In some embodiments, spinal correction system 20 may be employed with one or a plurality of vertebrae, for example, such that hooks 44, 52 of hook construct 28 span two or more vertebral levels, as described herein.

In use, to treat a selected section of vertebrae, which includes vertebrae V1-V4, a medical practitioner obtains access to a surgical site including vertebrae V1-V4 in any appropriate manner, such as through incision and retraction of tissues. In some embodiments, spinal correction system 20 can be used in any existing surgical method or technique including open surgery, mini-open surgery, minimally invasive surgery and percutaneous surgical implantation, whereby vertebrae V1-V4 is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal correction system 20. A preparation instrument (not shown) can be employed to prepare tissue surfaces of vertebrae V1-V4, as well as for aspiration and irrigation of a surgical region.

Construct 28 is delivered along the surgical pathway to a surgical site that includes vertebrae V1-V4. Construct 28 is delivered adjacent vertebra V2, such as, for example, an upper instrumented vertebra (UIV). Hook 44 is oriented with the bony anatomy of vertebra V2 such that the arcuate inner surface of hook 44 is oriented in an up-going configuration to engage a bottom or lower portion of a lamina of vertebra V2. Post 50 is selectively translated along passageway 32, in the direction shown by arrows B, BB in FIG. 3, such that the inner surface of hook 52 is oriented in a down-going configuration to engage a top or upper portion of the lamina of vertebra V2, in opposing relation to the surface of hook 44. Hook 52 may be rotated, about the axis of post 50, relative to body 29 to orient hook 52 for engagement with vertebral tissue. The arcuate inner surface of hook 44 maintains engagement with the lamina of vertebra V2 and construct 28 captures vertebra V2. In some embodiments, hooks 44, 52 may be disposed to selectively capture various portions of vertebral tissue, such as, for example, in a pedicle hook up-going, lamina capture, a transverse process down-going capture and/or combinations thereof. The threads of setscrew 42 are aligned with the threads of recess 40 and setscrew 42 is rotated to engage the threads of setscrew 42 to fix hook 44 relative to hook 52 and construct 28 with vertebra V2.

In one embodiment, spinal correction system 20 includes a bone fastener 80 including a receiver and a threaded shaft configured to penetrate tissue, such as, for example, bone. The receiver includes a pair of spaced apart arms that define a U-shaped implant cavity therebetween configured for disposal of a spinal construct, such as, for example, rod 22. Bone fastener 80 is delivered adjacent vertebra V4. The threaded shaft of bone fastener 80 is oriented with the bony anatomy of vertebra V4 and is manipulable to drive, torque, insert or otherwise connect bone fastener 80 with V4.

Rod 22 is delivered along the surgical pathway to the surgical site adjacent vertebrae V1-V4. In some embodiments, rod 22 and construct 28 and/or bone fastener 80 can be delivered or implanted as pre-assembled components or can be assembled in situ. Rod 22 is positioned for disposal within cavity 34 of construct 28 and the implant cavity of bone fastener 80 such that end 24 is disposed in cavity 34 of construct 28 and intermediate portion 25 is disposed in the implant cavity of bone fastener 80. In one embodiment, this configuration of the spinal construct allows the length of rod 22 to span upwardly and extend across only the vertebral level of vertebra V3, such as, for example, UIV-1, which is the level below upper instrumented vertebrae, and not across vertebra V2. In one embodiment, this configuration maintains mid-line structures intact to minimize disruption of an interface between vertebra V2 and V1. In one embodiment, this configuration transitions stiffness of the spinal construct and/or bone fastener 80 to the UIV. In some embodiments, rod 22 may be attached with vertebrae V with a plurality of bone fasteners 80 over a plurality of vertebral levels.

Setscrews are torqued and threaded with threads of cavity 34 of construct 28 and the implant cavity of bone fastener 80 to securely attach rod 22 with vertebrae V. The setscrews fix rod 22 relative to construct 28 and bone fastener 80 to prevent rod 22 from moving relative to construct 28 and/or bone fastener 80. In one embodiment, end 26 (FIG. 1) is attached to selected vertebra and/or vertebrae of vertebrae V spaced from vertebrae V1-V4.

In some embodiments, spinal correction system 20 may include a spinal construct comprising one or a plurality of constructs 28 that are each attachable with a selected vertebral level of vertebrae V, or two or more selected vertebral levels of vertebrae V. In one embodiment, spinal correction system 20 includes a second construct 28 (for example, as shown in FIG. 1) that is delivered adjacent vertebra V4, in addition to construct 28 that is delivered to vertebra V2, as described above. Hook 44 of the second construct 28 is oriented with the bony anatomy of vertebra V4 such that the arcuate inner surface of hook 44 of the second construct 28 is oriented in a down-going configuration to engage a top or upper portion of a lamina of vertebra V4. Post 50 of the second construct 28 is selectively translated along passageway 32 of the second construct 28 such that the inner surface of hook 52 of the second construct 28 is oriented in a down-going configuration to engage a bottom or lower portion of the lamina of vertebra V4, in opposing relation to the surface of hook 44 of the second construct 28. The arcuate inner surface of hook 44 of the second construct 28 maintains engagement with the lamina of vertebra V4 and the second construct 28 captures vertebra V4. Rod 22 is positioned for disposal within cavity 34 of the second construct 28 such that end 26 is disposed in cavity 34 of the second construct 28. Setscrews are torqued and threaded with threads of cavity 34 of the second construct 28 to securely attach rod 22 with vertebrae V, as described herein.

In some embodiments, spinal correction system 20 includes a spinal construct comprising hook construct 28 that is employed with a tether attached to a first side, such as, for example, a convex side of a spine that has a spinal disorder. In one embodiment, the tether and/or a spinal rod, may be affixed to the convex side of each of a plurality of vertebrae such that spinal correction system 20 prevents growth of vertebrae of a selected section of the spine while allowing for growth and adjustments to a second side, such as, for example, a concave side of the plurality of vertebrae for a correction treatment to treat various spine pathologies, such as, for example, adolescent idiopathic scoliosis and Scheuermann's kyphosis.

In some embodiments, all or only a portion of the tether may have flexible properties, such as the flexible properties corresponding to the material examples described above, such that the tether provides a selective amount of expansion and/or extension in an axial direction. The tether can include a plurality of separately attachable or connectable portions or sections, such as bands or loops, or may be monolithically formed as a single continuous element.

In some embodiments, the components of spinal correction system 20 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of spinal correction system 20 may be used to prevent or minimize curve progression in individuals of various ages.

In one embodiment, spinal correction system 20 includes an agent, which may be disposed, packed, coated or layered within, on or about the components and/or surfaces of spinal correction system 20. In some embodiments, the agent may include bone growth promoting material, such as, for example, bone graft to enhance fixation of the components and/or surfaces of spinal correction system 20 with vertebrae. In some embodiments, the agent may include one or a plurality of therapeutic agents and/or pharmacological agents for release, including sustained release, to treat, for example, pain, inflammation and degeneration.

Upon completion of the procedure, the surgical instruments, assemblies and non-implanted components of spinal correction system 20 are removed and the incision is closed. Spinal correction system 20 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal correction system 20. In some embodiments, spinal correction system 20 may include one or a plurality of plates, connectors and/or bone fasteners for use with a single vertebral level or a plurality of vertebral levels.

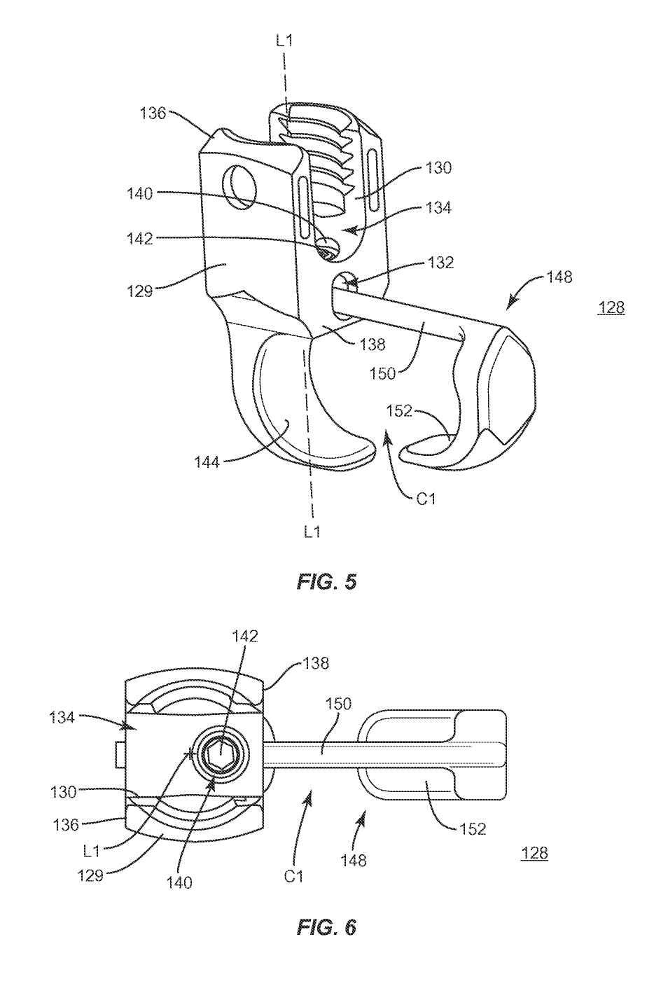

In one embodiment, as shown in FIGS. 5 and 6, spinal correction system 20, similar to the systems and methods described with regard to FIGS. 1-4, includes a member, such as, for example, a hook construct 128, similar to construct 28, which defines a longitudinal axis L1. Construct 128 includes a first surface, such as, for example, a body 129 including an inner surface 130. Body 129 includes a pair of arms that define a U-shaped cavity 134 extending parallel to axis L1 and configured for disposal of rod 22. At least a portion of cavity 134 is threaded and engageable with a coupling member, such as, for example, a setscrew to fix rod 22 relative to construct 128.

Construct 128 includes a passageway 132 having a closed configuration positioned distal to cavity 134 and extending parallel to axis L1. Passageway 132 is positioned distal to cavity 134 such that passageway 132 is spaced apart from cavity 134. Passageway 132 and cavity 134 each extend between and through an end surface 136 and an opposite end surface 138. Surface 130 defines a circular recess 140 extending parallel to axis L1 through cavity 134 and into passageway 132. Recess 140 includes a thread form configured to engage a thread form of a coupling member, such as, for example, a setscrew 142 to fix setscrew 142 within recess 140. Recess 140 is offset such that recess 140 is disposed closer to end 138 than end 136.

Construct 128 includes a hook 144, similar to hook 44 described herein, and an extension 148, similar to extension 48 described herein. Extension 148 includes a post 150 translatable within passageway 132 and a hook 152. Hook 152 is movable relative to hook 144 to define an adjustable tissue cavity C1 for connection with a portion of tissue, such as, for example vertebral tissue, similar to tissue cavity C described herein. In operation, a practitioner manipulates construct 128 for disposal about a vertebral level to fix and/or attach construct 128 with a targeted section of a spine for connecting rod 22 with vertebrae, similar to that described with regard to FIG. 4.

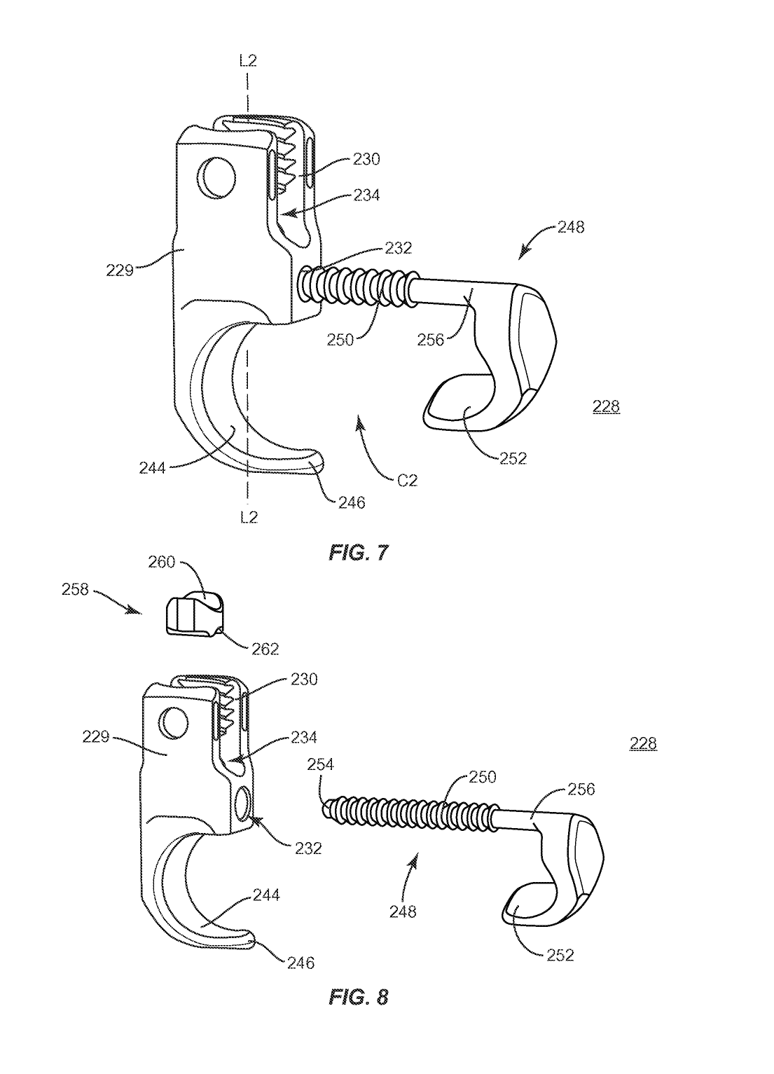

In one embodiment, as shown in FIGS. 7-9, spinal correction system 20, similar to the systems and methods described with regard to FIGS. 1-4, system 20 includes a member, such as, for example, a hook construct 228, similar to constructs 28, 128, which defines a longitudinal axis L2. Construct 228 includes a first surface, such as, for example, a body 229 including an inner surface 230. Body 229 includes a pair of arms that define a U-shaped cavity 234 extending parallel to axis L2 and configured for disposal of rod 22. Cavity 234 is threaded and engageable with a coupling member, such as, for example, a setscrew to fix rod 22 with construct 228.

Construct 228 includes a cylindrical passageway 232 extending perpendicular to axis L2. Passageway 232 is spaced apart from cavity 234. Passageway 232 and cavity 234 each extend between and through an end surface 236 and an opposite end surface 238. In some embodiments, passageway 232 is beveled adjacent surface 236 and/or surface 238 such that a width of passageway 232 increases adjacent surface 236 and/or surface 238 to facilitate insertion of a second surface, such as, for example, an extension 248 into passageway 232, in the direction shown by arrow H and relative to body 229.

Surface 230 defines a substantially circular recess 240 extending parallel to axis L2 in communication with cavity 234 and passageway 232. Recess 240 is configured for disposal of an insert, such as, for example, a crown 258 configured to facilitate locking of construct 228 about vertebral tissue, such as, for example, a vertebral level. Recess 240 is positioned equidistant between end 238 and end 236. In some embodiments, recess 240 is disposed closer to end 236 than end 238. In some embodiments, recess 240 is disposed closer to end 238 than end 236. In some embodiments, recess 240 is variously shaped, such as, for example, those alternative described herein.

Construct 228 includes a hook 244 having an inner surface having an arcuate configuration. The inner surface of hook 244 is oriented in a first direction such that the inner surface of hook 244 faces toward end 238. Hook 244 extends laterally from body 229 and includes a tip 246 that extends beyond surface 238.

Crown 258 includes a concave upper surface 260 and a concave lower surface 262. Surface 260 is disposed in substantial alignment with surface 230 when crown 258 is disposed within recess 240 such that surfaces 230, 260 define an implant cavity configured for disposal of rod 22. Surface 262 includes a plurality of gear teeth 264 configured to engage teeth 266 of a post 250 of extension 248 to selectively fix post 250 relative to insert 258. In some embodiments, all or only a portion of crown 258 is fabricated from a pliable, low-friction material, such as, for example, silicone, polyurethane, silicone-polyurethane copolymers, polymeric rubbers, polyolefin rubbers, elastomers, rubbers, thermoplastic elastomers, thermoset elastomers and elastomeric composites. In some embodiments, all or only a portion of crown 258 is fabricated from a semi-rigid, rigid or elastic configuration, relative to other components of construct 228 and/or have elastic properties, such as the elastic properties corresponding to the material examples described above.

Construct 228 includes extension 248 including post 250 and a hook 252 extending from post 250. Post 250 is translatable and/or rotatable within passageway 232 relative to body 229 and has a length extending between an end 254 and an end 256. Hook 252 extends from end 256 and includes an arcuate inner surface oriented in a second direction that faces toward the inner surface of hook 244. End 254 includes gear teeth 266 that engage teeth 264 for fixation of extension 248 in a selected position and/or orientation relative to body 229. Teeth 264, 266 engage to lock the surfaces of hooks 244, 252 with vertebral tissue, such as, for example, in compression about a lamina, transverse process and/or pedicle of a vertebral level.

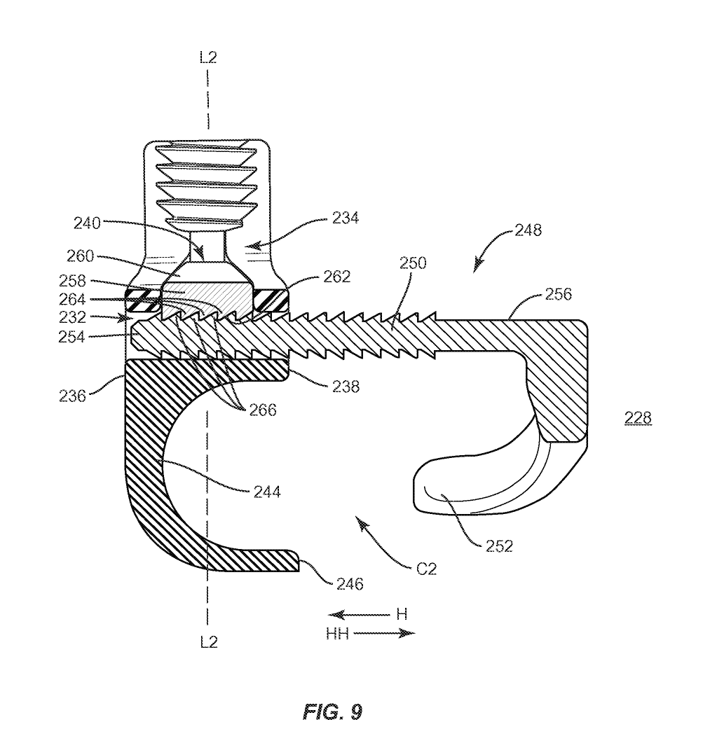

In one embodiment, teeth 264, 266 are relatively configured in a one way ratchet configuration such that post 250 is movable relative to insert 258, in the direction shown by arrow H in FIG. 9, for translating extension 248 relative to body 229. The one way ratchet configuration of teeth 264, 266 prevent post 250 from moving relative to crown 258, in the direction shown by arrow HH, such that extension 248 will not translate relative to body 229 in that direction.

Post 250 is translatable relative to crown 258 such that hook 252 translates relative to hook 244. As such, the inner surfaces of hooks 244, 252 define an adjustable tissue cavity C2, similar to cavity C described herein, for connection with a portion of tissue, such as, for example, vertebral tissue. In operation, a practitioner manipulates construct 228 for disposal about a vertebral level to fix and/or attach construct 228 with a targeted section of a spine for connecting rod 22 with vertebrae, similar to that described with regard to FIG. 4.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal construct comprising:
a longitudinal member extending between a first end and a second end and including an intermediate portion; and
a construct member including a first surface and a second surface movable relative to the first surface in a configuration for connection with a first portion of vertebral tissue, the construct member defining an implant cavity configured for disposal of the first end.

2. A spinal construct as recited in claim 1, wherein the surfaces of the construct member are movable to define an adjustable tissue cavity.

3. A spinal construct as recited in claim 2, wherein the tissue cavity is selectively adjustable about a configuration of the first portion of the vertebral tissue.

4. A spinal construct as recited in claim 1, wherein at least one of the surfaces includes an arcuate configuration.

5. A spinal construct as recited in claim 1, wherein the construct member comprises a first hook including the first surface oriented in a first direction and a second hook including the second surface oriented in a second direction.

6. A spinal construct as recited in claim 1, wherein the construct member comprises a body that includes the first surface and defines a passageway configured for disposal of the second surface of the construct member.

7. A spinal construct as recited in claim 6, further comprising a coupling member engageable with the body and the second surface of the construct member to selectively fix the second surface of the construct member relative to the body.

8. A spinal construct as recited in claim 6, wherein the passageway has an open configuration.

9. A spinal construct as recited in claim 6, wherein the passageway has a closed configuration.

10. A spinal construct as recited in claim 1, further comprising a coupling member engageable with the first surface of the construct member and the second surface of the construct member to selectively fix the second surface of the construct member relative to the first surface of the construct member.

11. A spinal construct as recited in claim 10, wherein the first surface of the construct member includes a body having an inner surface extending from a first end to a second end, the coupling member being centrally disposed with the inner surface.

12. A spinal construct as recited in claim 10, wherein the first surface of the construct member includes a body having an inner surface extending from a first end to a second end, the coupling member being disposed adjacent the first end of the inner surface.

13. A spinal construct as recited in claim 1, wherein the first surface of the construct member includes an inner surface having gear teeth, the second surface of the construct member including an outer surface having gear teeth, the gear teeth being engageable to selectively fix the second surface of the first member relative to the first surface of the construct member.

14. A spinal construct as recited in claim 13, wherein the inner surface includes an insert comprising the gear teeth of the first surface of the construct member.

15. A spinal construct as recited in claim 13, wherein the gear teeth comprise a single direction ratchet configuration to selectively fix the second surface of the construct member relative to the first surface of the construct member.

16. A spinal construct comprising:
a first hook construct comprising:
a body including an inner surface and an arcuate surface, the inner surface defining a passageway and a cavity for disposal of a first end of a spinal implant; and
an extension including a post and an arcuate surface, the post being translatable within the passageway such that the arcuate surface of the extension is movable relative to the arcuate surface of the body to selectively fix the body and the extension for capturing a first vertebral level.

17. A spinal correction system comprising:
a spinal rod extending between a first end and a second end and including an intermediate portion; and
a first hook construct including a first surface and a second surface movable relative to the first surface in a configuration for connection with a first vertebral level, the first hook construct defining an implant cavity configured for disposal of the first end.

18. A spinal correction system as recited in claim 17, further comprising:
a second hook construct including a first surface and a second surface movable relative to the first surface of the second hook construct in a configuration for connection with a second vertebral level spaced from the first vertebral level, the second hook construct defining an implant cavity configured for disposal of the spinal rod; and
a first coupling member engageable with the surfaces of the first hook construct to selectively fix the surfaces of the first hook construct about a configuration of the first vertebral level; and
a second coupling member engageable with the surfaces of the second hook construct to selectively fix the surfaces of the second hook construct about a configuration of the second vertebral level.

19. A spinal correction system as recited in claim 17, wherein:
a second hook construct including a first surface and a second surface movable relative to the first surface of the second hook construct in a configuration for connection with a second vertebral level spaced from the first vertebral level, the second hook construct defining an implant cavity configured for disposal of the intermediate portion,
the surfaces of the first hook construct each including an arcuate configuration, and
the surfaces of the second hook construct each including an arcuate configuration.

20. A spinal correction system as recited in claim 17, wherein the first surface of the first hook construct includes a body defining a passageway and the second surface of the first hook construct includes a post slidably translatable along the passageway.
